(19) United States
(12) Patent Application Publication
(10) Pub. No.: US 2010/0300451 A1
(43) Pub. Date: Dec. 2, 2010

(54) Punch Dilator

(76) Inventors: Nathan C. Griffith, Roswell, GA (US); Michael A. Kenowski, Alpharetta, GA (US); Sam C. Chan, Atlanta, GA (US); James F. Schumacher, Cumming, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/475,754

(22) Filed: Jun. 1, 2009

(51) Int. Cl. A61M 29/00; A61B 17/34

(52) U.S. Cl. 128/207.29

(57) ABSTRACT

There is provided a device for performing an initial piercing and dilating of a patient's trachea. The device has a needle within and extending beyond a sheath which in turn is within and extends beyond an introducer dilator. After the needle is used to pierce the trachea, it may be removed and a guide wire (J-wire) inserted through the introducer dilator and sheath in its place. The sheath is slidable within the introducer dilator once the needle is removed. The introducer dilator is then moved forward, into the site of the initial piercing to expand it. As the introducer dilator is moved forward, the sheath may remain stationary, sliding within the introducer dilator and thus reducing trauma to the stoma site.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through an ostomy in the tracheal wall. Endotracheal tubes have been linked in some studies to an increased rate of ventilator acquired pneumonia (VAP) and so tracheostomy operations are becoming increasingly common and are being performed earlier in the patient's hospital stay in order to reduce the occurrence of VAP.

A tracheostomy procedure involves making an incision in the skin of the neck to grant access to the trachea. Because of the uniquely flexible and elastic nature of the trachea, it has been found that healing is much faster if only a small hole is made in the trachea and the hole dilated, rather than cutting the trachea. After the skin incision, a hemostat or other implement may be used to separate the subcutaneous tissues to gain access to the trachea, and digital palpation is used to locate the tracheal rings. A bronchoscope is usually inserted into the ET tube and the tube withdrawn from the trachea until the light of the bronchoscope transdermally illuminates the site of the incision. A sheathed needle is used to puncture the trachea from the outside, usually between the second and third tracheal rings, the needle is removed with the sheath remaining, a flexible guide wire (also called a J-wire) is inserted in the place of the needle and then the sheath is removed. The bronchoscope is used for viewing the procedure from inside the trachea in order to avoid damage to the rear tracheal wall. A small (e.g. 14 French) initial dilator is introduced over the guide wire to perform an initial dilation of the trachea and the dilator is then removed. A smaller (e.g. 8 French) guiding catheter is then introduced over the guide wire. (Note, French is a measure of circumference based on the theory that non-round tubes of the same circumference will fit into the same incision. One French is approximately 0.33 mm or 0.013 inch).

After the guiding catheter is introduced, a larger, first dilator such as the Cook Medical Inc. Blue Rhino® dilator (see also U.S. Pat. No. 6,637,435), is placed over the guide wire and the guiding catheter and larger dilator are advanced into the trachea as a unit to perform the dilation. Cook Medical recommends a slight over-dilation in order to make the placement of the tracheostomy tube easier. After dilation, the larger dilator is removed and the tracheostomy tube (with cannula removed) is introduced over the guide catheter using a second dilator that fits just inside the trachostomy tube and protrudes about 2 cm beyond the distal end of the tracheostomy tube. The guide catheter, second dilator and tracheostomy tube are advanced into the trachea as a unit. Once the tracheostomy tube is at the proper depth, the second dilator, guide catheter and guide wire are removed through the tracheostomy tube, the inner cannula is inserted into the tracheostomy tube and the tube connected to the ventilator. The procedure is complete.

As can be understood from the above description, performing a tracheostomy involves numerous steps and the insertion and removal of a number of components before the successful completion of the procedure. For most of this time, the patient is disconnected from the ventilator and is therefore, not breathing. In addition, the large number of parts used in current tracheostomy kits increases the likelihood that an item may be accidentally rendered unsterile and be unable to be used. In such cases, the patient must be re-intubated with an ET tube. Even if the procedure proceeds uneventfully, however, the amount of time the patient is not breathing is significant; on the order of 7 minutes or more. This is clearly a significant event, especially for a patient who is, most likely, not in optimal physical condition.

There remains a need for a device that can more quickly and safely allow for the successful placement of a tracheostomy tube.

### SUMMARY

There is provided a punch dilator device that allows for the rapid puncture of the tracheal wall, insertion of a guide wire and initial dilation. The device has a needle within and extending beyond a sheath and introducer dilator. The needle may be detachably attached to the proximal end of the sheath and/or introducer dilator. The needle is used to pierce the trachea and is advanced far enough into the stoma site so that the sheath enters the stoma site. The needle may then be removed from the sheath and introducer dilator, leaving the sheath in the stoma site, and a guide wire (J-wire) inserted in place of the needle.

Once the needle is disconnected from the proximal end of the sheath and/or introducer dilator, the sheath may slide within the introducer dilator. The introducer dilator may be moved into the trachea to dilate the initial piercing or puncture created by the needle. The sheath may slide into the introducer dilator as the introducer dilator is moved into the trachea stoma site or the sheath may move forward, farther into the trachea. Should the sheath come in contact with the far tracheal wall, it may bend or move proximally back into the introducer dilator. Any procedure that involves placement of a dilator and subsequent dilation of a stoma would benefit from this novel device.

### BRIEF DESCRIPTION OF THE DRAWINGS

FIG. 1 is an exploded view of one embodiment of the punch dilator device showing the relationship of the components.

FIG. 2 is a drawing of the assembled punch dilator.

FIG. 3 is a cross-sectional view of the interior of the introducer dilator.

FIG. 4 depicts the punch dilator device after puncture of the trachea, with the needle attached.

FIG. 5 depicts the punch dilator with the needle removed and the sheath moved proximally into the dilator.

FIG. 6 depicts the punch dilator with the needle removed and the dilator moved into the stoma site to dilate the opening.

### DETAILED DESCRIPTION

Tracheostomy is a lifesaving procedure to allow a patient to be ventilated directly through the trachea. Tracheostomy is also believed by many to prevent or retard the onset of ventilator acquired pneumonia (VAP). This lifesaving procedure is, unfortunately, relatively time consuming and current technology requires a large number of steps and pieces of equipment that must remain sterile and functioning properly in order to arrive at a successful conclusion. This procedure may be greatly improved using the device described in the Summary above; the punch dilator (the device). In addition, the device may be used in emergency tracheotomies, and the term “tracheostomy” as used herein is meant to include the term tracheotomy.

The device replaces a number of pieces used in the procedure described in the introduction. The device replaces the separate needle, sheath and the introducer dilator and allows for the aspiration of the patient to ensure the needle has entered the trachea and not the esophagus or other tissue. The device is designed so that the procedure is, except of course for the initial piercing of the trachea, completely reversible at any point during the procedure. The body of the device allows for the reintroduction of the needle after it is removed, should that become necessary.

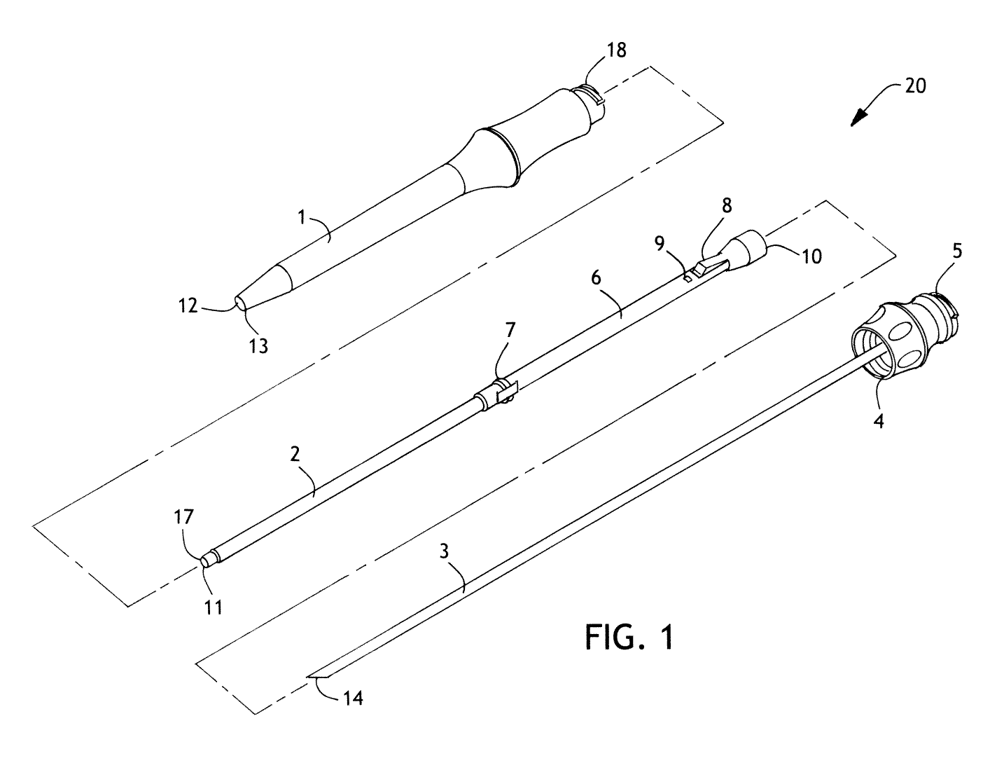

Turning to FIG. 1 in which an exploded view of one embodiment of the device 20 is shown, there are three primary components: a relatively more rigid, hollow, introducer introducer dilator 1, a more flexible inner sheath 2 having a lumen or canulla, and a needle 3. The needle 3 is more rigid than the sheath 2 and is preferably beveled on its distal end 14 and has a cutting edge to facilitate initial penetration of the intended stoma site on the patient's neck. The needle 3 is desirably hollow though it may be solid if insertion of a guidewire through the needle 3 is not desired. The needle 3 is attached to the center of a hub 4 that serves at least two purposes: it allows connection of a syringe via an integral standard ISO leur fitting 5, and it acts as a retainer to prevent the sheath 2, through which the needle 3 is inserted, from sliding within the introducer introducer dilator 1 while the needle is attached to the introducer introducer dilator 1.

The introducer introducer dilator 1 is shown in FIG. 1 as having an exterior surface that is more amenable to being gripped by the fingers, though the particular exterior shape is not meant to be limiting but merely a suggestion for possible embodiments. The exterior of the introducer dilator 1 may be textured for easier gripping as well. The introducer dilator 1 has an internal cavity extending along an axial dimension wherein the cavity is wider than the sheath 2 so as to allow the sheath 2 to move from side to side inside the introducer dilator 1 (without the needle present). The introducer dilator 1 has openings on both its distal and proximal ends that are large enough for the sheath 2 to pass through lengthwise (axially).

The sheath 2 has a canulla running its entire axial length so that the needle 3 may be inserted in the proximal end of the sheath 2 and, if the needle is of sufficient length, extend through the sheath 2 and out the distal end of the sheath 2 while part of the needle 3 still extends from the sheath 2 on the proximal end. The sheath 2 is generally flexible, meaning that it can bend within the introducer dilator 1 without kinking.

The needle 3 is releasably contained substantially within the cannula of the sheath 2 as explained above, and extends beyond the sheath 2 at the sheath's 2 distal and proximal ends. The needle 3 provides resistance to the movement of the sheath 2 when the needle 3 is positioned within the sheath 2, as will be explained in greater detail below. Removal of the needle 3 from the sheath 2 eliminates the resistance to movement of the sheath 2 within the introducer dilator 1 and allows the sheath 2 to slide within the introducer dilator 1 along the dilator's 1 axial dimension and to move from side to side within the dilator.

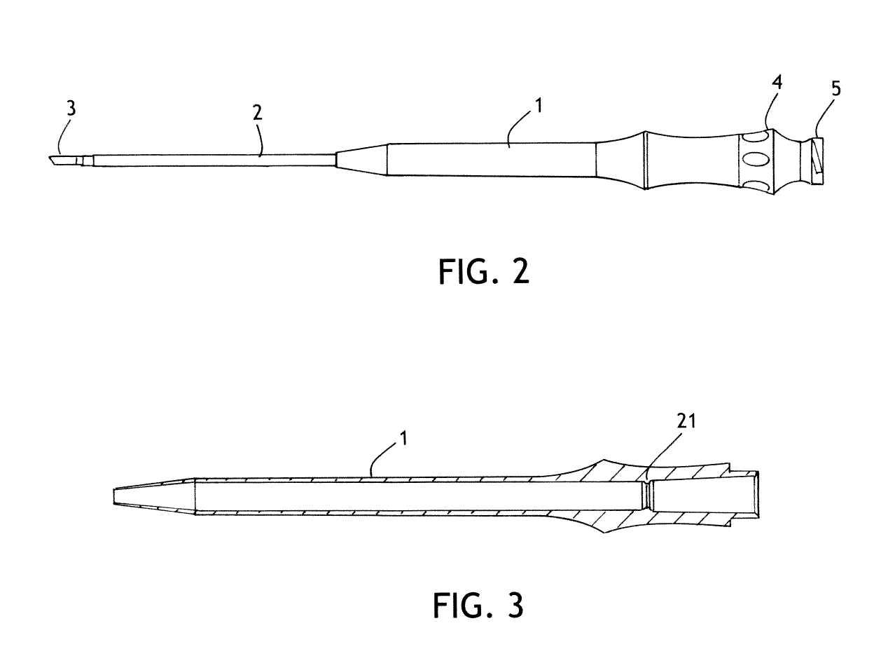

The hub 4 may be attached to the proximal end 18 of the introducer introducer dilator 1 by any satisfactory detachable means. In one embodiment, for example, threads are used for a screwed fitting, though leur, bayonet or other fittings may be used. In order to assemble the device, the needle 3 is inserted into the sheath 2 and then the sheath 2 and needle 3 are inserted into the introducer introducer dilator 1. The hub 4 is attached to the proximal end 18 of the introducer introducer dilator 1. This is the fully assembled state of the device 20 as shown in FIG. 2. The inner sheath 2 is then unable to move relative to either the needle 3 or the introducer introducer dilator 1 due to the role of the stop 8, (explained below) and the hub 4.

The sheath 2 in this embodiment includes an inner hub 6 with integral end stop 8. The needle hub 4 prohibits movement of the sheath 2 in the proximal direction by coming into contact with the inner dilator 2 at its proximal end 10. The proximal end stop 8 limits the movement of the inner sheath 2 in the distal direction by coming into contact with the interior of the introducer dilator 1 at its distal end 13. In this manner the sheath 2 always remains within the central cavity 12 of the introducer introducer dilator 1, and the distal tip 11 of the inner sheath 2 is prevented from overlapping the needle bevel 14 when the device 20 is fully assembled.

When the needle 3 is inserted into the sheath 2 and the sheath 2 is in turn inserted into the introducer introducer dilator 1, the hub 4 of the needle 3 acts to center the sheath 2 and needle 3 in the introducer dilator 1 since the needle 3 is connected to the center of the hub 4. When the needle 3 is withdrawn proximally from the sheath 2 after the trachea is punctured, as described above, the sheath 2 is prevented from also being withdrawn proximally by a bump 9. It is important that the distal tip 11 of the inner sheath 2 remains inside the introducer dilator 1 during withdrawal of the needle 3 to maintain the location for the guidewire 15, and the bump 9 prevents the inner sheath 2 from sliding axially when the needle 3 is removed. The bump 9 creates axial sliding resistance against a ring 21 on the inner surface of the introducer dilator 1 only when the sheath 2 is centered in the introducer dilator 1, i.e., only when the needle 3 is present. (FIG. 3) After the rigid needle 3 is withdrawn from the flexible sheath 2, the bump 9 will not be stopped by the ring 21 since the sheath 2 will now be allowed to bend slightly within the central cavity 12 and the bump 9 will slide by the ring 21, allowing the sheath 2 to travel proximally within the dilator. It should be noted, however, that the bump and ring arrangement is only one exemplary embodiment, and any other way of preventing the sheath from being withdrawn along with the needle would be satisfactory. As one example; a plurality of outwardly facing splines arranged around the circumference of the sheath and interacting against a ridge on the inside surface of the dilator could be used to prevent the withdrawal of the sheath with the needle.

As noted above, once the needle 3 is removed, the sheath 2 is not held in the center of the introducer dilator 1 but may bend from side to side withing the central cavity 12, and the bump 9 does not necessarily contact the ring 21, so the proximal movement of the sheath 2 in the introducer dilator 1 is limited only by the distal stop 7 coming into contact with the ring 21. The distal stop 7 may be somewhat chamfered to allow the insertion of the sheath 2 into the introducer dilator 1 for initial assembly, i.e. as the sheath 2 is inserted into the introducer dilator 1 the stop 7 slides by the ring 21. This is, however, a one-way process, and the interaction of the stop 7 and ring 21 prohibits the sheath 2 from moving proximally out of the introducer dilator 1, whether the needle 3 is present or not. Again, other systems to prevent the withdrawal of the sheath from the dilator may be devised and yet remain within the spirit and intention of the invention.

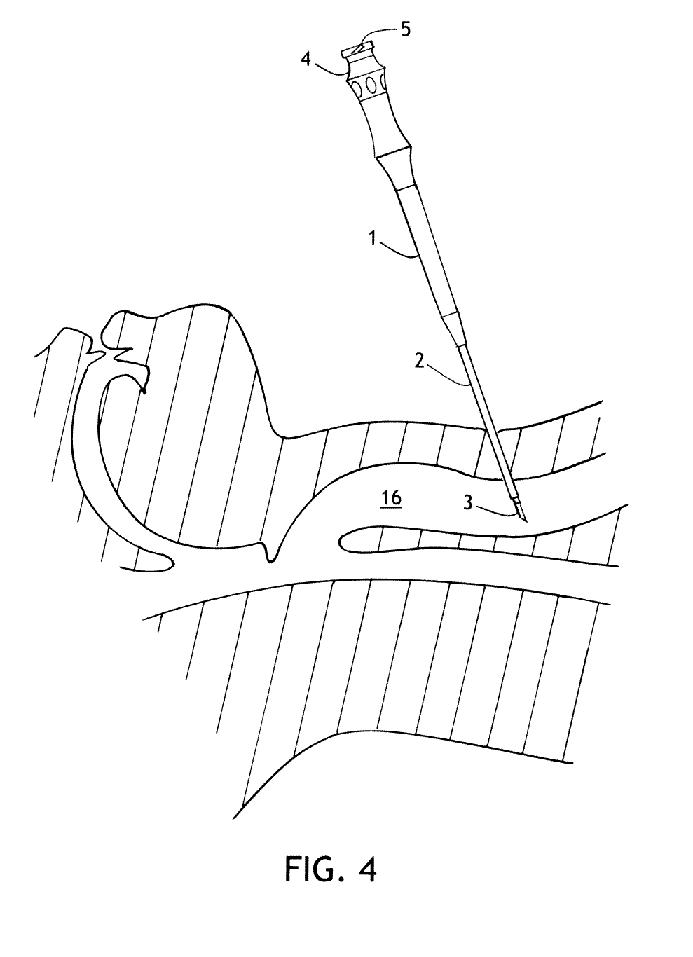

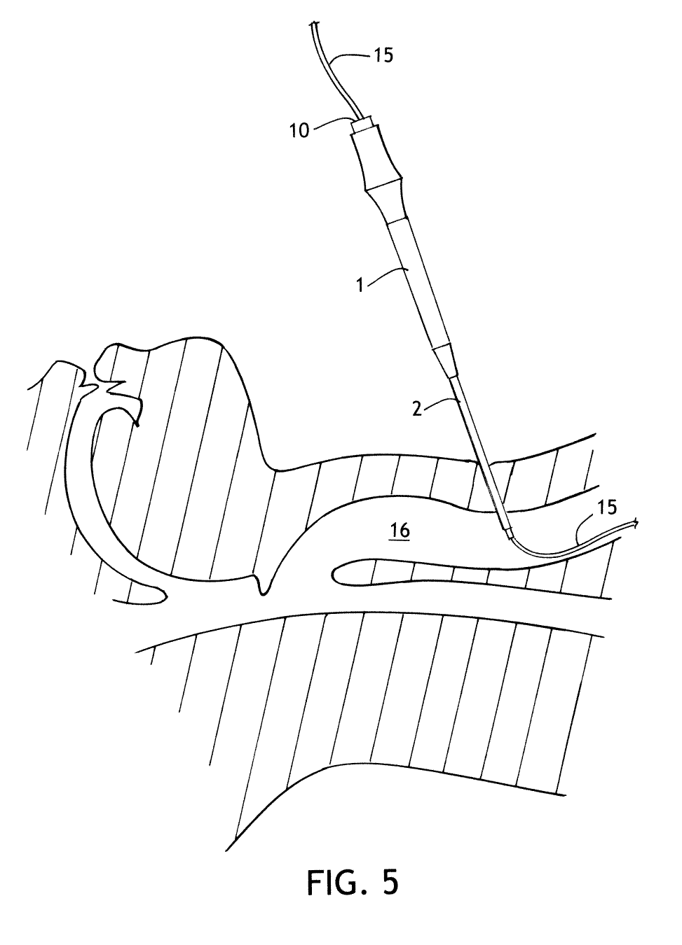

Once the needle 3 is used to puncture the neck at a stoma site and to enter the trachea, in embodiments using a hollow needle, a syringe (not shown) may be attached to the proximal end 19 of the hub 4 to aspirate a sample to ensure that the needle 3 has indeed entered the trachea 16 (FIG. 4). In this embodiment a standard leur fitting 5 is used to attach the syringe to the hub 4, though any other satisfactory means known to those skilled in the art may be used. Once it has been determined that the needle 3 has indeed entered the trachea 16, the needle 3 may be withdrawn from the trachea 16 and from the sheath 2 and introducer introducer dilator 1 by detaching the hub 4 from the introducer introducer dilator 1 and moving the hub 4 and attached needle 3 in the proximal direction until the needle 3 is withdrawn. The sheath 2 and introducer introducer dilator 1 remain in position with the sheath 2 partially in the trachea 16 stoma site as shown in FIG. 5. A guidewire 15 or “J-wire” may then be inserted in place of the needle 3 through the introducer introducer dilator 1 and sheath 2 into the trachea 16 (FIG. 5). The guidewire 15 is introduced to the trachea 16 through the central cannula or lumen 17 of the sheath 2 via the proximal opening 10 of the sheath 2. The proximal opening may be funnel shaped for ease of insertion of the needle 3 and guidewire 15, though there is no intention to limit the shape of the proximal opening 10 to any particular shape.

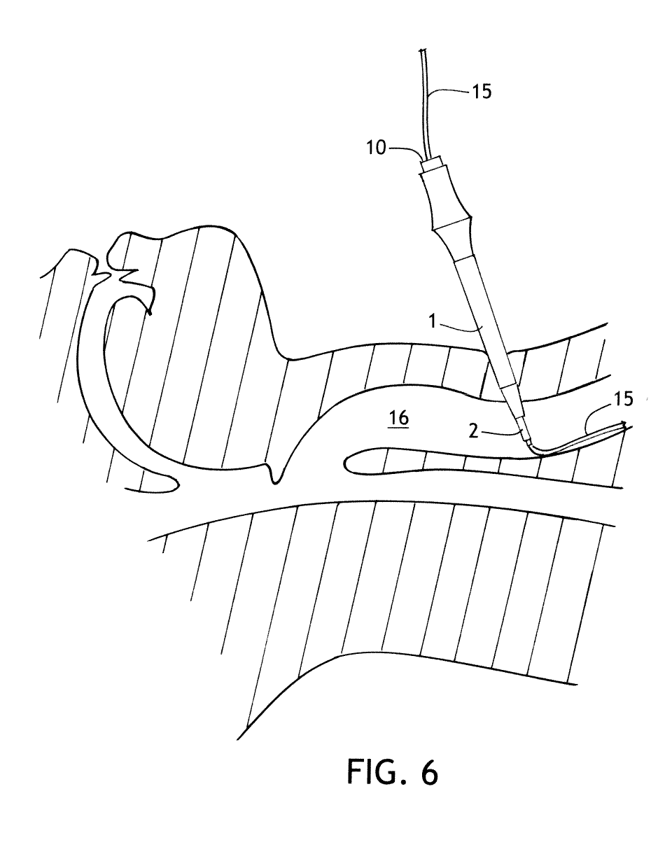

Once the guidewire 15 is in place, dilation of the stoma is achieved by moving the introducer introducer dilator 1 into the stoma site and, to some extent, the patient's trachea 16 (FIG. 6). The sheath 2 may remain stationary in relation to the stoma when the introducer introducer dilator 1 is inserted due to the freely sliding nature of their assembly, i.e., the sheath 1 will slide into the introducer dilator 1. This occurs up to the point where the distal stop 7 limits the movement of the sheath 2 relative to the introducer introducer dilator 1. It is believed that this feature of allowing the sheath 2 to remain stationary in the stoma site while moving the dilator forward until the dilator reaches the stoma site, may reduce trauma to the internal tissues of the patient caused by sliding the sheath 2 in the stoma site. Alternatively, the sheath 2 may slide further into the trachea as the introducer introducer dilator 1 is moved into the trachea 16. The sheath 2 may be made of a material that is relatively more flexible than the introducer introducer dilator 1 and so should bend if necessary should it come in contact with the back wall 18 of the trachea 16 as it is being inserted. Alternatively, the sheath 2 may be made of a material having the same flexibility as the introducer dilator 1 but may be made with thinner walls, thus affording the sheath 2 greater flexibility than the introducer dilator 1. After the stoma site is dilated, the balance of the device 20 is removed from the patient, leaving only the guidewire 15 in place to facilitate introduction of additional dilators if necessary.

At any time during the procedure, the steps outlined above may be reversed and the device 20 removed from the trachea. This allows great flexibility and control for the health care professional should there be an unforeseen complication that requires the reversal or immediate cessation of the procedure.

For ease of manufacture the components of the device 20 may be made as a number of separate parts and assembled to produce the final device 20. As can be seen in FIG. 1, parts of the device 10 are illustrated as separate components. The dashed lines are meant to indicate the assembly steps; needle 3 inserted into sheath 2 and then into introducer dilator 1. This is meant only as one means of or suggestion for producing the device and is not meant as a limitation or restriction of the disclosed concept.

The introducer dilator is desirably made of a relatively more rigid material since it is used to puncture the trachea. The relative hardness of the polymers used to make the dilator may be measured by the Shore hardness, a series of scales that is known to those skilled in the art. Hardness is measured using a device called a “durometer”, an instrument specifically developed to measure relative hardness, and is usually performed following ASTM D2240. In the Shore A and D hardness or durometer scales, a higher number indicates a polymer that is harder than a polymer having a lower number within each scale. The Shore A and D scales are used for different types of polymers. Typically the Shore A scale is used for softer, more elastic polymers and the Shore D scale used for stiffer polymers. When comparing the Shore A and Shore D scales, low D values are typically harder than high A values. For example, a 55D hardness is typically harder than a 90A shore hardness value. Desirably, the dilator may have a Shore hardness from 55D to 75D.

The materials of construction of the components of the device may be those commonly known to those skilled in the art. These include polyolefins, thermoplastic polyurethane elastomers, thermoplastic polyolefin elastomers, thermoplastic polyolefin block copolymers, SBS di-block elastomers, SEBS tri-block elastomers, polyvinyl chloride, polyethylene terephthalate and blends and mixtures thereof. A particularly suitable polymer is polyethylene. In one embodiment the dilator may be made from Marlex® 9018 high density polyethylene, available from Chevron Phillips Chemical Co. A suitable polymer for the sheath is BP Solvay's Fortiflex® high density polyethylene and the needle is typically made from 304 or 316 Stainless Steel.

Many of the features discussed in the embodiment above are optional and the punch dilator would function satisfactorily if they were not present. For example, the bump and ring arrangement need not be present or another system may be used to hold the sheath within the dilator when the needle is removed. Another system relying on the friction between the introducer sheath outer diameter and the introducer dilator distal tip inner diameter may be used as an alternate. The syringe connection may be omitted if desired. The needle hub may connect to the dilator in another way such as by simple tabs or by a luer or bayonet connection or the needle may simply be held in place during puncture of the trachea by the thumb or finger of the person performing the tracheostomy.

While the exact size of the punch dilator device may be varied, there are some recommended criteria that should be met. The device, for example, should have a total length of less than 25 cm, more particularly less than 18 cm and weigh less that about 20 grams, more particularly less than 10 gms. The device must be biocompatible, desirably free of di(2-ethylhexyl)phthalate (DEHP) and desirably free of animal derived products. The needle may be from 1 to 15 French, more particularly between 2 and 8 French and desirably about 4.5 French in size and should be the longest component of the device. The sheath is slightly larger than the needle; about 1 to 15 French, more particularly about 2 to 8 French, desirably about 6 French in size. The introducer dilator is from 5 to 20 French, more particularly between 11 and 18 French and desirably about 14 French and can be from about 40 to 70 mm long, more particularly between 45 and 65 mm and desirably about 50 to 55 mm long. The introducer dilator may be tapered at its distal end as visible in FIG. 1, to ease the transition from the sheath to the full outer diameter of the introducer dilator. It is recommended that the force required to separate the components of the device be not greater than 30 Newtons. The guide wire should be about 0.052 inches (0.020 cm) in diameter and pass through the device using a force of no more than 2 Newtons.

In one embodiment, the introducer dilator may be 86 mm long with the sheath extending an additional 45 mm beyond the end of the dilator and the needle extending an additional 5 mm beyond the end of the sheath. The needle may have an outside diameter of 1.5 mm and an internal diameter of 1.2 mm. The sheath may have an outside diameter of 2 mm and an internal diameter of 1.7 mm. The bump may extend 0.2 mm above the outside diameter of the sheath. The stops may extend 0.3 mm above the outside diameter of the sheath. The introducer dilator may have an internal diameter of 3.5 mm (not at the location of the ring) and an external diameter of 4.7 mm. The internal diameter of the introducer dilator at the ring may be 2.8 mm.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A punch dilator device comprising a needle, a sheath and an introducer dilator;
the sheath having a canulla running its entire axial length so that the needle may be inserted in a proximal end of the sheath, extend through the sheath and out a distal end of the sheath;
the sheath being inserted into the introducer dilator and extending distally beyond the end of the introducer dilator;
the introducer dilator having an internal cavity extending along an axial dimension, wherein the cavity is wider than the sheath so as to allow the sheath to move from side to side within the introducer dilator when the needle is not present, the introducer dilator having distal and proximal ends with openings large enough for the sheath to pass through lengthwise;
wherein the needle provides resistance to the movement of the sheath when the needle is positioned within the sheath in the introducer dilator and removal of the needle from the sheath and introducer dilator eliminates resistance to movement of the sheath within the introducer dilator and allows the sheath to slide within the introducer dilator along the introducer dilator's axial dimension and to move from side to side within the introducer dilator.

2. The device of claim 1 wherein said needle is detachably attached to a proximal end of the introducer dilator.

3. The device of claim 1 wherein said needle has a cannula.

4. The device of claim 3 wherein said cannula of said needle may be used to aspirate a patient after said needle is used to pierce a trachea.

5. The device of claim 1 wherein said needle is used to pierce the trachea at a stoma site and is inserted into said stoma site to a depth where said sheath is also in said stoma site, and said needle is then removed and replaced by a guide wire while said sheath remains in said stoma site.

6. The device of claim 5 wherein said introducer dilator is moved into said stoma site.

7. The device of claim 1 wherein said introducer dilator is between 5 to 20 French.

8. The device of claim 1 wherein said introducer dilator is 14 French.

9. The device of claim 1 wherein said needle is between 1 and 15 French.

10. A punch dilator device for performing a tracheostomy comprising an 11 to 18 French introducer dilator that is between 45 and 65 mm long, said introducer dilator surrounding a sheath that extends beyond the distal end of said introducer dilator and in which is located a removable 2 to 8 French needle having a cannula, wherein said needle extends beyond a distal end of said sheath.

11. The device of claim 10 wherein said cannula may be used to aspirate a patient.

12. The device of claim 10 wherein said needle is used to pierce a trachea and said needle is removed from said sheath and introducer dilator.

13. The device of claim 10 wherein said introducer dilator is releasably attached to said needle.
